(19) United States
(12) Patent Application Publication
(10) Pub. No.: US 2024/0245381 A1
(43) Pub. Date: Jul. 25, 2024

(54) BIOLOGICAL INFORMATION ACQUISITION DEVICE

(71) Applicant: Murata Manufacturing Co., Ltd., Nagaokakyo-shi (JP)

(72) Inventors: Shohei MORIKAWA, Nagaokakyo-shi (JP); Hirofumi WATANABE, Nagaokakyo-shi (JP); Kiyoshi KANAGAWA, Nagaokakyo-shi (JP); Kiyotaka ASAI, Nagaokakyo-shi (JP)

(21) Appl. No.: 18/598,309

(22) Filed: Mar. 7, 2024

Related U.S. Application Data
(63) Continuation of application No. PCT/JP2022/024005, filed on Jun. 15, 2022.

(30) Foreign Application Priority Data
Sep. 29, 2021 (JP) 2021-159265

(51) Int. Cl. A61B 7/04; A61B 5/00; A61B 5/0205; A61B 5/259; A61B 5/282; A61B 7/00

(52) U.S. Cl. CPC A61B 7/04; A61B 5/02055; A61B 5/259; A61B 5/282; A61B 5/6823; A61B 7/003; A61B 7/008; A61B 2560/0468; A61B 2562/0271

(57) ABSTRACT

A biological information acquisition device includes a main body including a facing surface facing a living body when mounted, a first biosensor provided in the main body such that at least a portion of the first biosensor protrudes from the facing surface of the main body and including a first contact surface contactable with the living body, and an adhesive tape attached to the facing surface of the main body and having a thickness less than a protrusion amount of the first biosensor from the facing surface.

### CROSS REFERENCE TO RELATED APPLICATIONS

This application claims the benefit of priority to Japanese Patent Application No. 2021-159265 filed on Sep. 29, 2021 and is a Continuation Application of PCT Application No. PCT/JP2022/024005 filed on Jun. 15, 2022. The entire contents of each application are hereby incorporated herein by reference.

### BACKGROUND OF THE INVENTION

### 1. Field of the Invention

The present disclosure relates to a biological information acquisition device that acquires a biological sound such as a lung sound (respiratory sound, adventitious sound, etc.) and a heart sound generated from a living body, as biological information.

### 2. Description of the Related Art

For example, Japanese Unexamined Patent Application Publication No. 2017-169648 discloses a biosensor that acquires a respiratory sound waveform, a heart sound waveform, an electrocardiographic waveform, and the like as biological information. This biosensor includes a substrate, a covering plate that covers the substrate, and a piezoelectric element that is disposed between the substrate and the covering plate to acquire a respiratory sound waveform.

### SUMMARY OF THE INVENTION

However, in a case of the biosensor described in Japanese Unexamined Patent Application Publication No. 2017-169648, a degree of close contact between a portion of the substrate provided with the piezoelectric element and the living body is low. Therefore, there is a case where the piezoelectric element cannot accurately acquire a respiratory sound waveform or a heart sound waveform.

Example embodiments of the present invention provide biological information acquisition devices each capable of improving an ability to make close contact with a living body to acquire biological information with high accuracy.

According to an aspect of an example embodiment of the present disclosure, a biological information acquisition device includes a main body including a facing surface facing a living body when mounted, a first biosensor provided in the main body such that at least a portion of the first biosensor protrudes from the facing surface of the main body and including a first contact surface contactable with the living body, a first rigid portion that supports the first biosensor, and a lid that is attachable to and detachable from the first rigid portion, in which the lid surrounds a periphery of the first biosensor and is attachable to and detachable from the first rigid portion by being rotated in a direction along the periphery of the first biosensor.

In addition, according to another aspect of an example embodiment of the present disclosure, a biological information acquisition device includes a main body including a facing surface that faces a living body when mounted, and a first biosensor that is provided in the main body and includes a first contact surface contactable with the living body, in which the main body includes a first rigid portion that supports the first biosensor, and a deformable portion that supports the first rigid portion and is softer than the first rigid portion.

According to example embodiments of the present disclosure, it is possible to provide biological information acquisition devices each capable of enhancing an ability to make close contact with a living body to acquire biological information with high accuracy.

The above and other elements, features, steps, characteristics and advantages of the present invention will become more apparent from the following detailed description of the example embodiments with reference to the attached drawings.

### BRIEF DESCRIPTION OF THE DRAWINGS

FIG. 1 is an upper perspective view of a biological information acquisition device according to an example embodiment of the present invention.

FIG. 2 is a lower perspective view of the biological information acquisition device.

FIG. 3 is a side view of the biological intonation acquisition device.

FIG. 4 is a top view of the biological information acquisition device.

FIG. 5 is an upper exploded perspective view of the biological information acquisition device.

FIG. 6 is a lower exploded perspective view of the biological information acquisition device.

FIG. 7 is a cross-sectional view of the biological information acquisition device taken along a line A-A in FIG. 4.

FIG. 8 is a cross-sectional view of the biological information acquisition device taken along a line B-B in FIG. 4.

FIG. 9 is a cross-sectional view of the biological information acquisition device taken along a line C-C in FIG. 4.

FIG. 10 is a block diagram of a control system of the biological information acquisition device.

FIG. 11 is a diagram showing the biological information acquisition device in a state of being mounted on a living body.

FIG. 12 is a perspective view of the biological information acquisition device showing another mounting mode on the living body.

FIG. 13 is a schematic bottom view of a biological information acquisition device according to another example embodiment of the present invention.

FIG. 14 is a schematic bottom view of a biological information acquisition device according to still another example embodiment of the present invention.

FIG. 15 is a schematic bottom view of a biological information acquisition device according to still another example embodiment of the present invention.

FIG. 16 is a schematic bottom view of a biological information acquisition device according to still another example embodiment of the present invention.

### DETAILED DESCRIPTION OF THE EXAMPLE EMBODIMENTS

A bio-acoustic sensor according to an aspect of an example embodiment of the present disclosure includes a main body including a facing surface facing a living body when mounted, a first biosensor provided in the main body such that at least a portion of the first biosensor protrudes from the facing surface of the main body and including a first contact surface contactable with the living body, a first rigid portion that supports the first biosensor, and a lid that is attachable to and detachable from the first rigid portion, in which the lid surrounds a periphery of the first biosensor, and is attachable to and detachable from the first rigid portion by being rotated in a direction along the periphery of the first biosensor.

According to this aspect, it is possible to provide a biological information acquisition device capable of enhancing an ability to make close contact with a living body to acquire biological information with high accuracy.

For example, the first biosensor may be a bio-acoustic sensor that includes a vibration plate including the first contact surface and of which at least a portion protruding from the facing surface of the main body, and a piezoelectric element to detect vibration of the vibration plate and to measure a sound emitted from the living body.

For example, the biological information acquisition device may further include a second biosensor that is provided in the main body such that at least a portion of the second biosensor protrudes from the facing surface and includes a second contact surface contactable with the living body, and a distance from the facing surface to the first contact surface of the first biosensor may be larger than a distance from the facing surface to the second contact surface of the second biosensor.

For example, the second biosensor may be an electrocardiographic sensor that includes a plurality of electrodes each including the second contact surface and operable to acquire an electrocardiographic waveform of the living body, and the plurality of electrodes of the second biosensor may be provided in the main body such that the first biosensor is located between the plurality of electrodes.

For example, the second contact surfaces of the plurality of electrodes of the second biosensor may come into contact with the living body with conductive gels interposed therebetween, and the plurality of electrodes may be provided in the main body such that surfaces of the conductive gels that come into contact with the living body are located on substantially the same plane as the first contact surface of the first biosensor.

For example, the main body may include a first rigid portion that supports the first biosensor, a plurality of second rigid portions that support the plurality of respective electrodes of the second biosensor, and a detainable portion that supports the first rigid portion and the plurality of second rigid portions and is softer than the first rigid portion and the plurality of second rigid portions.

For example, the first biosensor may protrude more than the plurality of second rigid portions from the facing surface of the main body to a living body side.

For example, the biological information acquisition device may further include a temperature sensor to acquire a body temperature of the living body, and the temperature sensor may acquire the body temperature by using at least one of the plurality of electrodes.

According to another aspect of an example of the present disclosure, a biological information acquisition device includes a main body including a facing surface that faces a living body when mounted, and a first biosensor that is provided in the main body and includes a first contact surface contactable with the living body, in which the main body includes a first rigid portion that supports the first biosensor, and a detainable portion that supports the first rigid portion and is softer than the first rigid portion.

According to this aspect, it is possible to provide a biological information acquisition device capable of enhancing an ability to make close contact with a living body to acquire biological information with high accuracy.

For example, the first biosensor may be a bio-acoustic sensor that includes a vibration plate including the first contact surface and a piezoelectric element to detect vibration of the vibration plate and to measure a sound emitted from the living body.

For example, the biological information acquisition device may further include a second biosensor including a second contact surface contactable with the living body, and the main body may include a second rigid portion that supports the second biosensor.

For example, the second biosensor may include an electrocardiographic sensor that includes a plurality of electrodes each including the second contact surface and operable to acquire an electrocardiographic waveform of the living body, a plurality of the second rigid portions maybe provided, and the plurality of electrodes of the second biosensor may be provided in the plurality of second rigid portions. The second electrode is not limited to a rectangular shape, and may have a circular shape or other shape.

For example, the first rigid portion may be between the plurality of second rigid portions.

For example, the first rigid portion may be spaced apart from each of the plurality of second rigid portions.

Hereinafter, example embodiments of the present disclosure will be described with reference to the drawings.

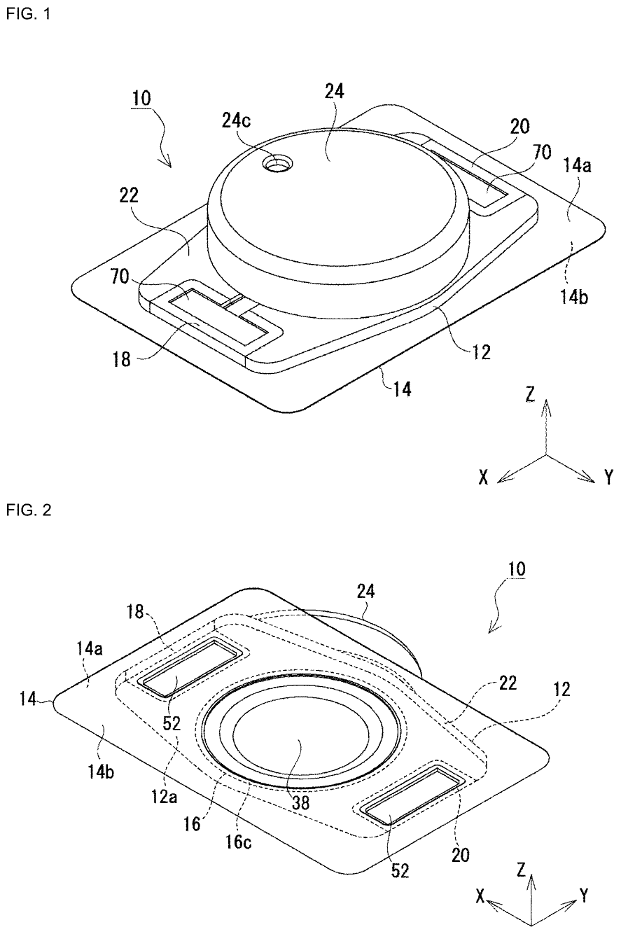

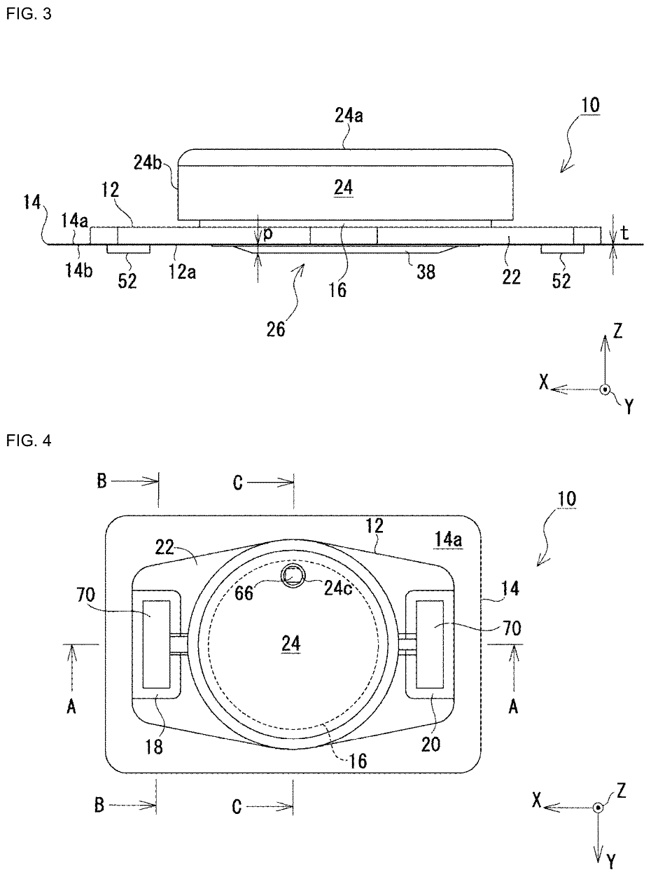

FIG. 1 is an upper perspective view of a biological information acquisition device according to an example embodiment of the present disclosure. In addition, FIG. 2 is a lower perspective view of the biological information acquisition device. Further, FIG. 3 is a side view of the biological information acquisition device. Furthermore, FIG. 4 is a top view of the biological information acquisition device. An X-Y-Z orthogonal coordinate system shown in the drawing is intended to facilitate understanding of the example embodiments of the present disclosure, and does not limit the example embodiments. An X-axis direction indicates a longitudinal direction of the biological information acquisition device, a Y-axis direction indicates a transverse direction, and a Z-axis direction indicates a height direction.

A biological information acquisition device 10 according to the present example embodiment shown in FIG. 1 is a device that acquires a biological sound waveform generated from a living body as biological information.

In addition, in a case of the present example embodiment, the biological information acquisition device 10 is configured to acquire a biological sound waveform, an electrocardiographic waveform, and a body temperature as the biological information.

As shown in FIGS. 1 to 4, in a case of the present example embodiment, a main body 12 of the biological information acquisition device 10 is mounted on the living body by using an adhesive tape 14.

Specifically, as shown in FIG. 2, the main body 12 of the biological information acquisition device 10 has a plate shape and includes a facing surface 12a that faces the living body when mounted. In a case of the present example embodiment, the adhesive tape 14 is a double-sided tape, and the facing surface 12a of the main body 12 is adhered to one adhesive surface 14a. The other adhesive surface 14b of the adhesive tape 14 is attached to the living body. That is, the facing surface 12a of the main body 12 of the biological information acquisition device 10 is attached to the living body by using the adhesive tape 14. The adhesive tape 14 may include a first tape of which one surface is a weakly viscous adhesive surface attached to the living body and the other surface is a smooth surface, and a second tape of which one surface is a strongly viscous adhesive surface attached to the other surface of the first tape and the other surface is a strongly viscous adhesive surface attached to the main body 12.

In a case of the present example embodiment, the main body 12 of the biological information acquisition device 10 includes a first rigid portion 16 and second rigid portions 18 and 20 that are substantially not defamed, and a detainable portion 22 that supports the rigid portions 16, 18, and 20 and is detainable.

In a case of the present example embodiment, the first rigid portion 16 and the second rigid portions 18 and 20 in the main body 12 of the biological information acquisition device 10 are made of a substantially non-deformable material such as a hard resin. On the other hand, the deformable portion 22 is made of a material softer (deformable) than the rigid portions, such as a soft resin, rubber, or cloth. In other words, the deformable portion 22 is more easily defamed than the first rigid portion 16 and the second rigid portions 18 and 20. Here, the tem. “soft” or “easily deformable” means that an elastic modulus such as a Young's modulus or a shear modulus is relatively small.

According to the configuration of the main body 12, the facing surface 12a of the main body 12 of the biological information acquisition device 10 can be brought into close contact with the living body with a high degree of close contact as compared to a case where the main body 12 is made only of a non-deformable material such as a hard resin. That is, even when a shape of a skin surface of the living body changes, the detainable portion 22 is defamed accordingly, so that the facing surface 12a of the main body 12 can continue to be in close contact with the living body without a gap. The detainable portion 22 is defamed, so that an impact on the main body 12 can be reduced even when the adhesive tape is peeled off and falls.

In the main body 12 of the biological information acquisition device 10, the first rigid portion 16 is disposed at a center of the main body 12 in a longitudinal direction (X-axis direction). The second rigid portions 18 and 20 are disposed to face each other in the longitudinal direction with the first rigid portion 16 interposed therebetween. That is, the first rigid portion 16 is disposed between the second rigid portions 18 and 20. In addition, in a case of the present example embodiment, the first rigid portion 16 is spaced apart from each of the second rigid portions 18 and 20. In addition, a portion interposed between the rigid portions 16, 18, and 20 is the deformable portion 22. In addition, the second rigid portions 18 and 20 are provided at both ends of the main body 12 in the longitudinal direction, respectively. In this manner, the rigid portions are spaced apart from each other so that the main body 12 is easily defamed.

A plurality of elements included to acquire biological information are provided in each of the first rigid portion 16 and the second rigid portions 18 and 20 in the main body 12 of the biological information acquisition device 10.

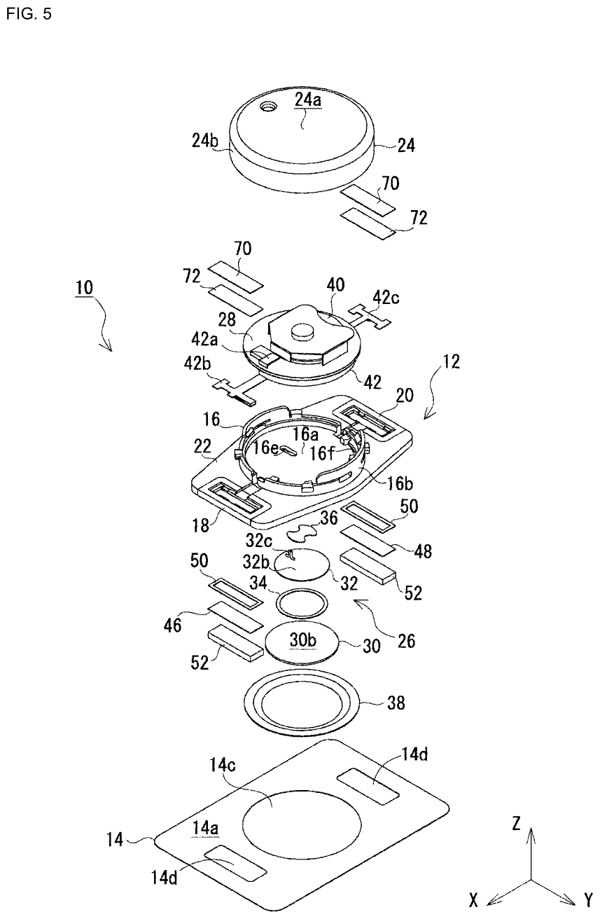

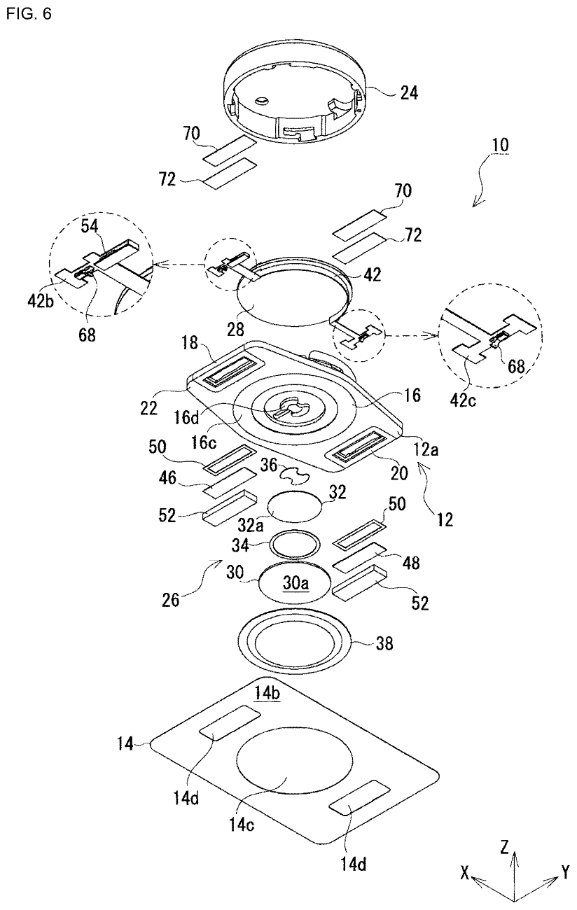

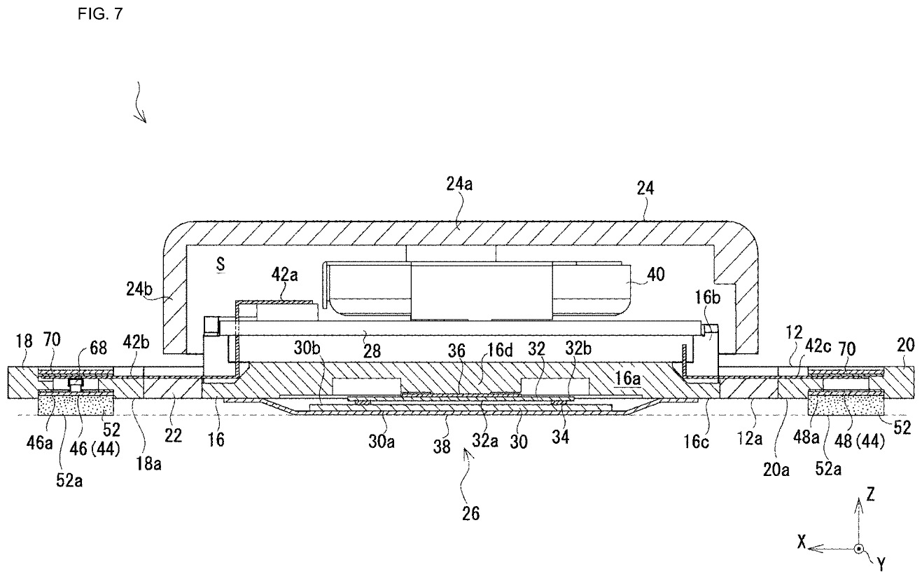

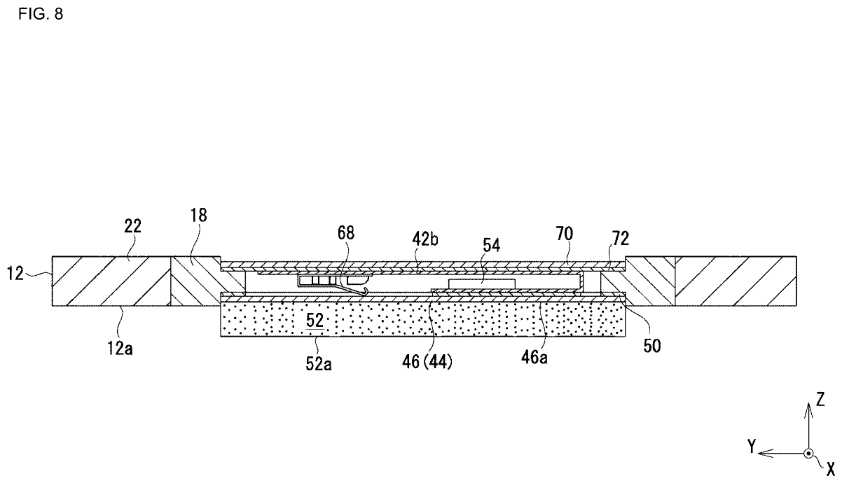

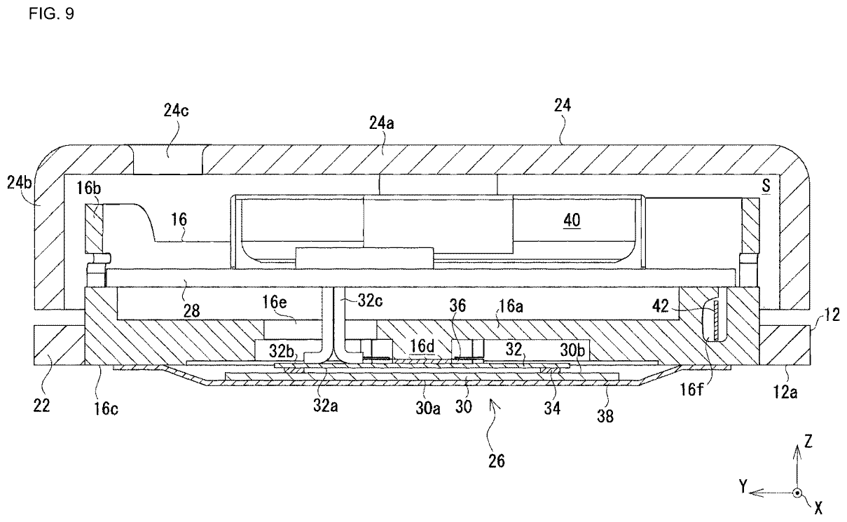

FIG. 5 is an upper exploded perspective view of the biological information acquisition device. In addition, FIG. 6 is a lower exploded perspective view of the biological information acquisition device. Further, FIG. 7 is a cross-sectional view of the biological information acquisition device taken along a line A-A in FIG. 4. Furthermore, FIG. 8 is a cross-sectional view of the biological information acquisition device taken along a line B-B in FIG. 4. FIG. 9 is a cross-sectional view of the biological information acquisition device taken along a line C-C in FIG. 4.

As shown in FIGS. 5 and 9, in a case of the present example embodiment, the first rigid portion 16 has a substantially bottomed cylindrical shape, and includes a bottom plate portion 16a and an annular wall portion 16b that extends in the height direction (Z-axis direction) from an outer peripheral edge of the bottom plate portion 16a. In a case of the present example embodiment, an outer surface 16c of the bottom plate portion 16a of the first rigid portion 16 defines a portion of the facing surface 12a of the main body 12.

In addition, a lid 24 is attached to the first rigid portion 16 in a detachable manner. As shown in FIG. 9, the lid 24 includes a top plate portion 24a that faces the bottom plate portion 16a of the first rigid portion 16 at a distance, and an annular wall portion 24b that extends in the height direction (Z-axis direction) from an outer peripheral edge of the top plate portion 24a to surround the annular wall portion 16b of the first rigid portion 16. In a case of the present example embodiment, the lid 24 is fixed to the first rigid portion 16 by being rotated in one rotation direction about a rotation center line extending in the height direction. When the lid 24 is rotated in the other rotation direction, the lid 24 is in a state of being removable from the first rigid portion 16.

As shown in FIG. 9, the first rigid portion 16 is provided with a bio-acoustic sensor 26 as a first biosensor and a control board 28.

The bio-acoustic sensor 26 is a sensor that acquires a biological sound waveform of a living body, and an element thereof is incorporated in the bottom plate portion 16a of the first rigid portion 16.

In a case of the present example embodiment, as shown in FIGS. 5 and 6, the bio-acoustic sensor 26 includes a disk-shaped vibration plate 30 having a contact surface 30a contactable with a living body, and a disk-shaped piezoelectric element 32 that detects vibration of the vibration plate 30. The vibration plate 30 and the piezoelectric element 32 are not limited to a disk shape, and may have other shapes such as a square shape and a rectangular shape.

Further, in a case of the present example embodiment, as shown in FIGS. 5, 6, and 9, the bio-acoustic sensor 26 includes an annular vibration transmitter 34 that is disposed between the vibration plate 30 and the piezoelectric element 32 and connects the vibration plate 30 and an outer peripheral edge portion of the first surface 32a of the piezoelectric element 32. The piezoelectric element 32 is attached to a top surface of a bulge portion 16d of the first rigid portion 16 that is bulged toward a living body side, by using a double-sided tape 36, at a central portion of the second surface 32b. A signal line 32c of the piezoelectric element 32 passes through a through hole 16e in the bottom plate portion 16a of the first rigid portion 16 and is connected to the control board 28. As a result, when the vibration plate 30 vibrates, the entire piezoelectric element 32 bends and detains, and the piezoelectric element 32 can detect the vibration with high resolution.

In a case of the present example embodiment, a thin film sheet 38 that covers and protects the bio-acoustic sensor 26 is attached to the outer surface 16c of the first rigid portion 16. Therefore, the contact surface 30a of the vibration plate 30 comes into contact with the living body with the film sheet 38 interposed therebetween.

In a case of the present example embodiment, the bio-acoustic sensor 26 is provided in the main body 12, that is, the first rigid portion 16 such that at least a portion of the bio-acoustic sensor 26 protrudes from the facing surface 12a of the main body 12. Specifically, at least a portion of the vibration plate 30 of the bio-acoustic sensor 26 protrudes from the facing surface 12a of the main body 12. The contact surface 30a is provided at a distal end of the protruding portion of the vibration plate 30. As a result, the contact surface 30a of the bio-acoustic sensor 26 (vibration plate 30 thereof) and the living body are in close contact with each other with a high close contact ability, and the bio-acoustic sensor 26 can acquire the vibration (biological sound) generated from the living body with high accuracy.

Specifically, when the facing surface 12a of the main body 12 is in close contact with the living body with the adhesive tape 14 interposed therebetween, the contact surface 30a of the vibration plate 30 of the bio-acoustic sensor 26 protruding from the facing surface 12a is strongly in close contact with the living body (as compared to a case where the bio-acoustic sensor 26 does not protrude from the facing surface 12a). Accordingly, the vibration plate 30 vibrates as if in synchronization with the vibration of the living body. The piezoelectric element 32 can detect the vibration of the living body with high accuracy via the vibration plate 30.

Furthermore, in a case of the present example embodiment, as described above, the main body 12 of the biological information acquisition device 10 includes the detainable portion 22. Therefore, even when the shape of the skin surface of the living body changes, the detainable portion 22 is defamed accordingly, so that the facing surface 12a of the main body 12 can continue to be in close contact with the living body without a gap. As a result, the bio-acoustic sensor 26 can continuously detect the vibration of the living body while maintaining high accuracy.

In a case of the present example embodiment, the bio-acoustic sensor 26 is provided not in the deformable portion 22 of the main body 12 but in the first rigid portion 16. As a result, vibration of a portion of the living body in contact with the bio-acoustic sensor 26 is transmitted to the bio-acoustic sensor 26 with a small loss.

On the other hand, in a case where the bio-acoustic sensor 26 is provided in the deformable portion 22, a portion of vibration energy of the living body is used for the deformation of the deformable portion 22, and the vibration transmitted to the bio-acoustic sensor 26 is attenuated. As a result, vibration detection accuracy of the bio-acoustic sensor 26 is reduced. Therefore, the bio-acoustic sensor 26 is provided not in the deformable portion 22 but in the first rigid portion 16.

In addition, in a case of the present example embodiment, as shown in FIG. 3, the adhesive tape 14 is attached to the facing surface 12a of the main body 12 of the biological information acquisition device 10. Therefore, a protrusion amount p of the bio-acoustic sensor 26 from the facing surface 12a of the main body 12 is larger than a thickness t of the adhesive tape 14. That is, the adhesive tape 14 has a thickness t smaller than the protrusion amount p of the bio-acoustic sensor 26. As shown in FIGS. 5 and 6, a through hole 14c through which the bio-acoustic sensor 26 passes is famed in the adhesive tape 14. A cutout may be famed in the adhesive tape 14 instead of the through hole 14c. That is, the adhesive tape 14 is disposed to avoid (not to overlap in a plan view) the bio-acoustic sensor 26.

As shown in FIG. 9, the control board 28 is accommodated in a space S defined by the first rigid portion 16 and the lid 24. In a case of the present example embodiment, the control board 28 is a circular circuit board, and a battery 40 is mounted on one surface thereof. The battery 40 is replaceable by removing the lid 24 from the first rigid portion 16. In addition, as shown in FIGS. 5 and 6, a flexible printed circuit board 42 is connected to the control board 28. Details of the control board 28 and the flexible printed circuit board 42 will be described later.

As shown in FIG. 7, a second biosensor 44 is provided in each of the second rigid portions 18 and 20. Specifically, the second biosensor 44 is an electrocardiographic sensor that acquires an electrocardiographic waveform of a living body, and each of a plurality of electrodes 46 and 48 that come into contact with the living body is provided in the second rigid portions 18 and 20. As shown in FIGS. 5 and 6, the electrodes 46 and 48 are attached to the second rigid portions 18 and 20 by using annular double-sided tapes 50.

In order to acquire a good electrocardiographic waveform, it is preferable that the plurality of electrodes 46 and 48 of the electrocardiographic sensor 44 are separated from each other. Therefore, in the biological information acquisition device 10, the plurality of electrodes 46 and 48 are provided in the main body 12 such that the bio-acoustic sensor 26 is located between the electrodes 46 and 48. As a result, the biological information acquisition device 10 is made small while the plurality of electrodes 46 and 48 are separated from each other as much as possible.

As shown in FIG. 7, the plurality of electrodes 46 and 48 of the electrocardiographic sensor 44 include contact surfaces 46a and 48a that come into contact with a living body, respectively. In a case of the present example embodiment, the contact surfaces 46a and 48a are located on a side opposite to the living body with respect to the facing surface 12a of the main body 12. As a result, the contact surface 30a of the bio-acoustic sensor 26 is located on a living body side as compared to the contact surfaces 46a and 48a.

Therefore, in a case of the present example embodiment, as shown in FIG. 7, conductive gels 52 are attached to the contact surfaces 46a and 48a of the plurality of electrodes 46 and 48. As a result, the contact surfaces 46a and 48a come into contact with the living body with the conductive gel 52 interposed therebetween. The plurality of electrodes 46 and 48 are provided in the main body 12 such that surfaces 52a of the conductive gels 52 that come into contact with the living body are located substantially on the same plane as the contact surface 30a of the bio-acoustic sensor 26. That is, a distance from the facing surface 12a of the main body 12 to the surface 52a of the conductive gel 52 (that is, a distance in the height direction (Z-axis direction)) and a distance from the facing surface 12a to the contact surface 30a of the bio-acoustic sensor 26 are equal to each other. Accordingly, the contact surface 30a of the bio-acoustic sensor 26 can be brought into close contact with the living body. In a case of the present example embodiment, as shown in FIGS. 5 and 6, a through hole 14d through which the conductive gel 52 passes is famed in the adhesive tape 14. A cutout may be famed in the adhesive tape 14 instead of the through hole 14d. That is, the adhesive tape 14 is disposed to avoid (not to overlap in a plan view) the conductive gel 52.

In a case where the contact surfaces 46a and 48a of the electrodes 46 and 48 come into direct contact with the living body without the conductive gels 52 interposed therebetween, a distance from the facing surface 12a of the main body 12 to the contact surfaces 46a and 48a (that is, a distance in the height direction (Z-axis direction)) is preferably smaller than a distance from the facing surface 12a to the contact surface 30a of the bio-acoustic sensor 26. Accordingly, the contact surface 30a of the bio-acoustic sensor 26 can be brought into close contact with the living body.

Furthermore, as shown in FIG. 7, in order for the contact surface 30a of the bio-acoustic sensor 26 to be in close contact with the living body, the bio-acoustic sensor 26 preferably protrudes more than the plurality of second rigid portions 18 and 20 from the facing surface 12a of the main body 12 to the living body side. In a case of the present example embodiment, surfaces 18a and 20a of the second rigid portions 18 and 20 facing the living body define a portion of the facing surface 12a of the main body 12. Accordingly, the contact surface 30a of the bio-acoustic sensor 26 can be brought into close contact with the living body.

In a case of the present example embodiment, as shown in FIG. 8, the biological information acquisition device 10 includes a temperature sensor 54 to detect a temperature of the electrode 46. The temperature sensor 54 indirectly detects a body temperature of the living body by detecting the temperature of the electrode 46 in contact with the living body. Here, the body temperature is a temperature of the skin surface. The body temperature is a core body temperature as long as the temperature sensor is capable of measuring the core body temperature. In addition, the temperature sensor 54 is connected to an electrode other than the electrodes 46 and 48, and can measure the body temperature when in contact with the living body. For example, the temperature sensor 54 may be provided in the bio-acoustic sensor 26.

The electrocardiographic sensor 44 acquires an electrocardiographic waveform of a living body based on a change in a potential difference between the plurality of electrodes 46 and 48.

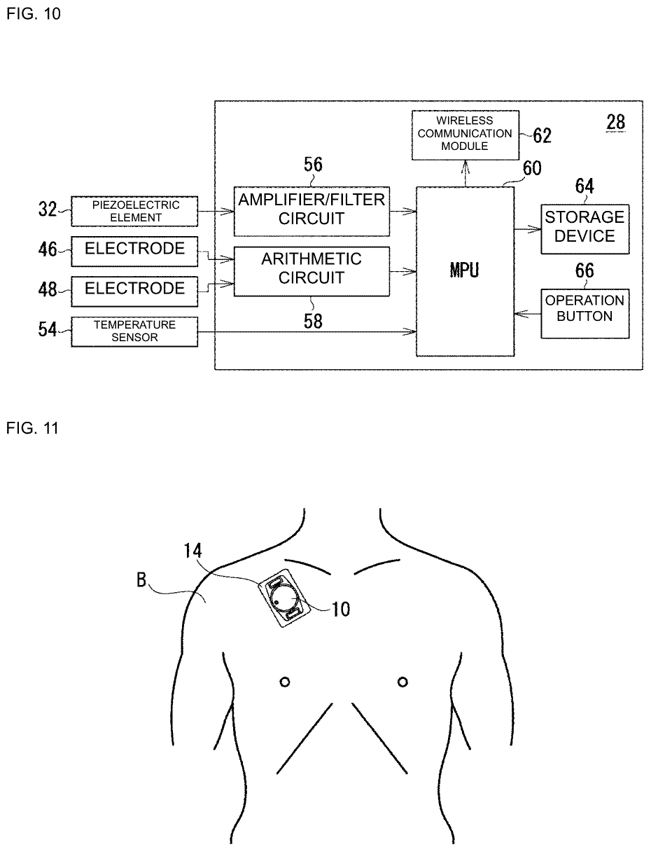

FIG. 10 is a block diagram of a control system of the biological information acquisition device.

As shown in FIG. 10, the control board 28 of the biological information acquisition device 10 is provided with an amplifier/filter circuit 56 to amplify and filter an output value (voltage signal) from the bio-acoustic sensor 26 (piezoelectric element 32). In addition, the control board 28 is provided with an arithmetic circuit 58 that calculates an electrocardiographic waveform based on a potential difference between the plurality of electrodes 46 and 48, as an element of the electrocardiographic sensor 44.

The biological sound waveform from the bio-acoustic sensor 26 processed by the amplifier/filter circuit 56 is subjected to analog/digital conversion (A/D conversion) by a microprocessor unit (MPU) 60 provided on the control board 28. Similarly, the electrocardiographic waveform calculated by the arithmetic circuit 58 and the body temperature from the temperature sensor 54 are subjected to A/D conversion by the MPU 60. The MPU 60 may include a CPU, a memory, various circuits, and the like to execute various processes.

Biological sound waveform data, electrocardiographic waveform data, and body temperature data created by the A/D conversion by the MPU 60 are transmitted to an external device via a wireless communication module 62 provided on the control board 28. In addition, these data are stored in a storage device 64 such as a memory provided on the control board 28. The wireless communication module 62 is a wireless communication module that complies with a wireless communication standard such as Bluetooth, and transmits the biological sound waveform data, the electrocardiographic waveform data, and the temperature data to, for example, a mobile terminal. In a case where the biological information acquisition device 10 includes an output module such as a display that can output the biological sound waveform data and the like, and/or in a case where the biological information acquisition device 10 includes a writer module that writes data to a storage medium such as a memory card, the wireless communication module can be omitted.

The control board 28 is provided with an operation button 66 that starts or stops the acquisition of the biological sound waveform, the electrocardiographic waveform, and the body temperature. As shown in FIG. 4, the operation button 66 is operated through a through hole 24c famed in the top plate portion 24a of the lid 24.

As shown in FIGS. 5 to 8, the plurality of electrodes 46 and 48 of the electrocardiographic sensor 44 and the temperature sensor 54 are connected to the control board 28 via the flexible printed circuit board 42. The flexible printed circuit board 42 includes a first connection end 42a connected to the control board 28, a second connection end 42b connected to the electrode 46, and a third connection end 42c connected to the electrode 48. The temperature sensor 54 is mounted on the second connection end 42b of the flexible printed circuit board 42. The plurality of electrodes 46 and 48 are electrically connected to the flexible printed circuit board 42 via spring terminals 68 provided at the second and third connection ends 42b and 42c of the flexible printed circuit board 42. The manufacturing of the biological information acquisition device 10 is facilitated by electrical connection via the spring terminal 68 as compared to a case of electrical connection via solder. In addition, the electrodes 46 and 48 are urged toward the living body by the spring terminals 68, and the close contact ability between the living body and the electrodes 46 and 48 is improved.

The plurality of electrodes 46 and 48 of the electrocardiographic sensor 44 are in contact with the spring terminals 68 of the flexible printed circuit board 42 in the second rigid portions 18 and 20 of the main body 12. As a result, the contact is maintained. On the contrary, when the electrodes 46 and 48 and the spring terminal 68 come into contact with each other in the deformable portion 22 of the main body 12, the contact may be released due to the defamation of the deformable portion 22. Therefore, the electrodes 46 and 48 are in contact with the spring terminals 68 in the second rigid portions 18 and 20 that are substantially not defamed. Rigid cover plates 70 covering the second and third connection ends 42b and 42c of the flexible printed circuit board 42 in the second rigid portions 18 and 20 are attached to the second rigid portions 18 and 20 and the second and third connection ends 42b and 42c by using double-sided tapes 72. The cover plate 70 functions as a retainer that receives a reaction force of the spring terminal 68.

As shown in FIGS. 5 and 9, in the flexible printed circuit board 42, a groove 16f that extends along the annular wall portion 16b and accommodates a portion of the flexible printed circuit board 42 (a portion extending on a rear side of the control board 28) is famed in the bottom plate portion 16a of the first rigid portion 16. By accommodating a portion of the flexible printed circuit board 42 in the groove 16f, a size of the biological information acquisition device 10 in the height direction (Z-axis direction) can be reduced.

Next, a usage method of the biological information acquisition device 10 will be described.

FIG. 11 shows the biological intonation acquisition device in a state of being mounted on a living body, as an example.

As shown in FIG. 11, the biological information acquisition device 10 is attached to a body B of the living body by using the adhesive tape 14. When the operation button 66 is pressed, the biological information acquisition device 10 starts to acquire the biological sound waveform, the electrocardiographic waveform, and the body temperature as the biological information, and transmits these data to the external device via the wireless communication module 62. The acquisition of the biological information may be started after, for example, a switch provided in the mobile terminal is operated in addition to the operation button 66. In FIG. 11, the biological information acquisition device 10 is attached to the vicinity of the clavicle of the body, but is not limited thereto, and may be attached to the abdomen, the back, the neck, or the like according to the biological information to be acquired.

According to the present example embodiment as described above, it is possible to provide a biological information acquisition device capable of enhancing the ability to make close contact with a living body to acquire biological information with high accuracy.

Although the present disclosure has been described above with reference to the plurality of example embodiments, the example embodiments of the present disclosure are not limited thereto.

For example, in a case of the above-described example embodiments, the biological information acquisition device 10 acquires a lung sound waveform, the electrocardiographic waveform, and the body temperature as the biological information. However, the present example embodiment is not limited thereto. For example, the bio-acoustic sensor may measure other biological sounds emitted from the living body, such as a heart sound waveform or an intestinal peristalsis sound. That is, the biological information acquisition devices according to the example embodiments of the present disclosure may be a device contactable with a living body to acquire biological information thereof.

In addition, in a case of the above-described example embodiments, the electrocardiographic sensor 44 acquires the electrocardiographic waveform using two electrodes 46 and 48. However, the number of the electrodes is not limited to two. For example, in a case of acquiring a 3-lead electrocardiographic waveform, the electrocardiographic sensor includes three electrodes.

Further, in a case of the above-described example embodiments, as shown in FIGS. 2 and 3, the facing surface 12a of the main body 12 of the biological information acquisition device 10 is attached to the living body by using the adhesive tape 14 having adhesive surfaces on both surfaces. However, the present example embodiments are not limited thereto.

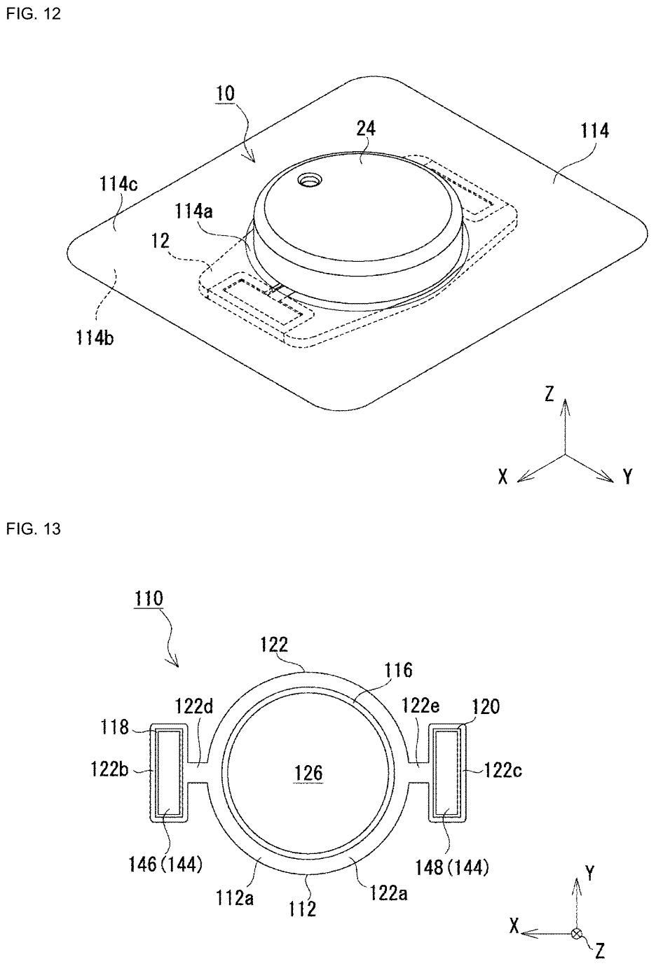

FIG. 12 is a perspective view of the biological information acquisition device showing another mounting mode on a living body.

As shown in FIG. 12, the biological information acquisition device 10 is attached to the living body by using an adhesive tape 114. The adhesive tape 114 includes a through hole 114a through which the lid 24 of the biological information acquisition device 10 passes. In addition, one surface 114b of the adhesive tape 114 is an adhesive surface, and the other surface 114c is a smooth surface. A portion of the one surface 114b, which is the adhesive surface, is attached to the main body 12 of the biological information acquisition device 10, and a remaining portion is attached to the living body. Instead of providing the through hole 114a, the adhesive tape 114 may cover the entire biological information acquisition device 10. In addition, although not shown, the biological information acquisition device may be fixed to the living body by a band wound around the living body, a garment worn on the living body, or the like, instead of the adhesive tape.

In addition, in a case of the above-described example embodiment, as shown in FIG. 4, a contour shape of the main body 12 of the biological information acquisition device 10, that is, a contour shape of the detainable portion 22 is substantially rectangular when viewed in the height direction (Z-axis direction) of the biological information acquisition device 10. However, the example embodiments of the present disclosure are not limited thereto.

FIG. 13 is a schematic bottom view of a biological information acquisition device according to another example embodiment of the present disclosure.

As shown in FIG. 13, in a biological information acquisition device 110 according to another example embodiment, a detainable portion 122 of a main body 112 has a minimum required shape. Specifically, the detainable portion 122 includes a central portion 122a that holds a first rigid portion 116 provided with a bio-acoustic sensor 126, and outer portions 122b and 122c that hold second rigid portions 118 and 120 provided with electrodes 146 and 148 of electrocardiographic sensor 144, respectively. In addition, the deformable portion 122 includes a strip-shaped connecting portion 122d that connects the central portion 122a and the outer portion 122b to each other, and a strip-shaped connecting portion 122e that connects the central portion 122a and the outer portion 122c to each other. The sizes of the strip-shaped connecting portions 122d and 122e in the transverse direction (Y-axis direction) of the biological information acquisition device 110 are smaller than the sizes of the central portion 122a and the outer portions 122b and 122c. Therefore, the outer portions are more easily displaced with respect to the central portion of the main body 112, that is, the main body 112 is more easily defamed. As a result, the facing surface 112a of the main body 112 is easily brought into close contact with the living body.

Further, in a case of the above-described example embodiment, as shown in FIGS. 5 and 6, the electrodes 46 and 48 of the electrocardiographic sensor 44 have a rectangular shape when viewed in the height direction (Z-axis direction) of the biological information acquisition device 10. However, the example embodiments of the present disclosure are not limited thereto.

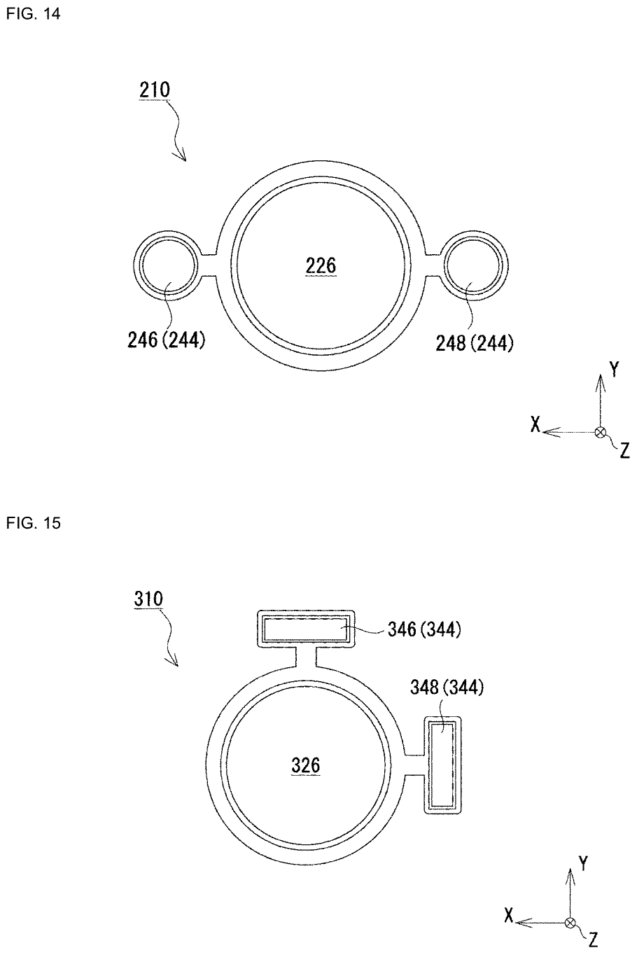

FIG. 14 is a schematic bottom view of a biological information acquisition device according to still another example embodiment of the present disclosure.

As shown in FIG. 14, in a biological information acquisition device 210 according to still another example embodiment, electrodes 246 and 248 of an electrocardiographic sensor 244 have a circular shape when viewed in the height direction (Z-axis direction). That is, the electrodes 246 and 248 have a shape similar to that of a bio-acoustic sensor 226. The shape of the electrode of the electrocardiographic sensor may be a shape other than a rectangular shape or a circular shape.

Furthermore, in a case of the above-described example embodiment, as shown in FIG. 7, when viewed in the height direction (Z-axis direction) of the biological information acquisition device 10, the bio-acoustic sensor 26 and the electrodes 46 and 48 of the electrocardiographic sensor 44 are arranged in a line in the longitudinal direction (X-axis direction), and the bio-acoustic sensor 26 is disposed between the electrodes 46 and 48. That is, the electrodes 46 and 48 are disposed such that angular positions thereof with respect to the bio-acoustic sensor 26 are different by 180 degrees. However, the example embodiments of the present disclosure are not limited thereto.

FIG. 15 is a schematic bottom view of a biological information acquisition device according to still another example embodiment of the present disclosure.

As shown in FIG. 15, in a biological information acquisition device 310 according to still another example embodiment, a bio-acoustic sensor 326 and electrodes 346 and 348 of an electrocardiographic sensor 344 are not arranged in a line. Instead, the electrodes 346 and 348 are disposed such that angular positions thereof with respect to the bio-acoustic sensor 326 are different by 90 degrees. As described above, the angular positions of the electrodes 346 and 348 with respect to the bio-acoustic sensor 326 may be other angular positions. However, in order to acquire a good electrocardiographic waveform, it is preferable that two electrodes of the electrocardiographic sensor are separated from each other.

In addition, in a case of the above-described example embodiment, in the biological information acquisition device 10, the bio-acoustic sensor 26 and the electrocardiographic sensor 44 are provided in a non-separable manner. However, the example embodiments of the present disclosure are not limited thereto.

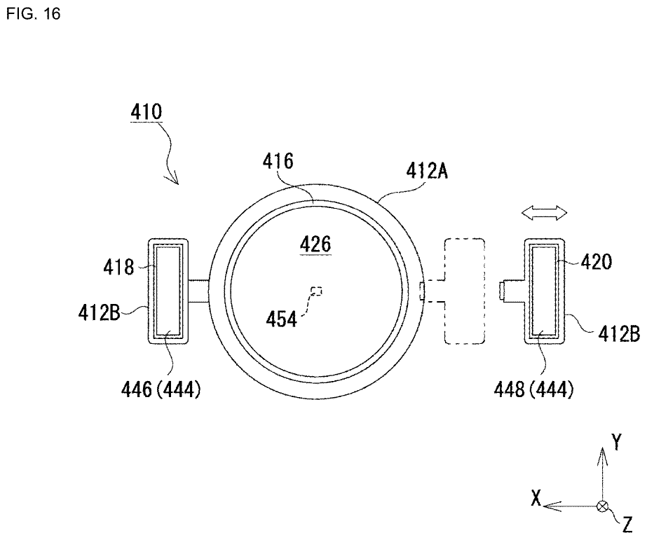

FIG. 16 is a schematic bottom view of a biological information acquisition device according to still another example embodiment of the present disclosure.

As shown in FIG. 16, in a biological information acquisition device 410 according to still another example embodiment, a main body thereof is configured to be separable into a main portion 412A including a first rigid portion 416, a bio-acoustic sensor 426, a control board, a battery, and the like, and an optional portion 412B including electrodes 446 and 448 of an electrocardiographic sensor 444, second rigid portions 418 and 420, a conductive gel, and the like. Accordingly, the main portion 412A can be reused, and the optional portion 412B can be disposable. Alternatively, a usage method thereof can be in reverse. In addition, in a case where the living body does not require electrocardiography, the biological sound can be acquired only by the main portion 412A. The main portion 412A and the optional portion 412B include a connector for electrically connecting to each other. In addition, in a case where the optional portion 412B is disposable, a temperature sensor 454 that measures the body temperature of the living body is provided in the main portion 412A. The temperature sensor 454 measures the body temperature, for example, via a vibration plate of the bio-acoustic sensor 426 that is in contact with the living body.

That is, a biological information acquisition device of an example embodiment according to an example embodiment of the present disclosure broadly includes a main body including a facing surface facing a living body when mounted, a first biosensor provided in the main body such that at least a portion of the first biosensor protrudes from the facing surface of the main body and including a first contact surface contactable with the living body, a first rigid portion that supports the first biosensor, and a lid that is attachable to and detachable from the first rigid portion, in which the lid surrounds a periphery of the first biosensor and is attachable to and detachable from the first rigid portion by being rotated in a direction along the periphery of the first biosensor.

In addition, a biological information acquisition device of another example embodiment of the present disclosure broadly includes a main body including a facing surface that faces a living body when mounted, and a first biosensor that is provided in the main body and includes a first contact surface contactable with the living body, in which the main body includes a first rigid portion that supports the first biosensor, and a deformable portion that supports the first rigid portion and is softer than the first rigid portion.

Example embodiments of the present disclosure are applicable to devices that are each able to come into close contact with a living body to acquire biological information of the living body.

While example embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A biological information acquisition device comprising:
a main body including a facing surface facing a living body when mounted;
a first biosensor provided in the main body such that at least a portion of the first biosensor protrudes from the facing surface of the main body and including a first contact surface contactable with the living body;
a first rigid portion that supports the first biosensor; and
a lid that is attachable to and detachable from the first rigid portion; wherein
the lid surrounds a periphery of the first biosensor, and is attachable to and detachable from the first rigid portion by being rotated in a direction along the periphery of the first biosensor.

2. The biological information acquisition device according to claim 1, wherein the first biosensor includes a bio-acoustic sensor that includes a vibration plate including the first contact surface and of which at least a portion protrudes from the facing surface of the main body, and a piezoelectric element to detect vibration of the vibration plate and to measure a sound emitted from the living body.

3. The biological information acquisition device according to claim 1, further comprising:
a second biosensor that is provided in the main body such that at least a portion of the second biosensor protrudes from the facing surface and includes a second contact surface contactable with the living body; wherein
a distance from the facing surface to the first contact surface of the first biosensor is larger than a distance from the facing surface to the second contact surface of the second biosensor.

4. The biological information acquisition device according to claim 3, wherein
the second biosensor includes an electrocardiographic sensor that includes a plurality of electrodes each including the second contact surface and operable to acquire an electrocardiographic waveform of the living body; and
the plurality of electrodes of the second biosensor are provided in the main body such that the first biosensor is located between the plurality of electrodes.

5. The biological information acquisition device according to claim 4, wherein
the second contact surfaces of the plurality of electrodes of the second biosensor are contactable with the living body with conductive gels interposed therebetween; and
the plurality of electrodes are provided in the main body such that surfaces of the conductive gels that are contactable with the living body are located on the same plane as the first contact surface of the first biosensor.

6. The biological intonation acquisition device according to claim 4, wherein the main body includes a first rigid portion that supports the first biosensor, a plurality of second rigid portions that support the plurality of respective electrodes of the second biosensor, and a deformable portion that supports the first rigid portion and the plurality of second rigid portions and is softer than the first rigid portion and the plurality of second rigid portions.

7. The biological intonation acquisition device according to claim 6, wherein the first biosensor protrudes more than the plurality of second rigid portions from the facing surface of the main body to a living body side.

8. The biological information acquisition device according to claim 4, further comprising:
a temperature sensor to acquire a body temperature of the living body; wherein
the temperature sensor is operable to acquire the body temperature by using at least one of the plurality of electrodes.

9. The biological information acquisition device according to claim 1, wherein the biological information acquisition device is structured and operable to detect a lung sound waveform, an electrocardiographic waveform, a heart sound waveform or an intestinal peristalsis sound.

10. The biological information acquisition device according to claim 4, wherein each of the plurality of electrodes has a rectangular or circular shape.

11. The biological information acquisition device according to claim 4, wherein angular positions of the plurality of electrodes with respect to the first biosensor are different by 90 or 180 degrees.

12. A biological information acquisition device comprising:
a main body including a facing surface that faces a living body when mounted; and
a first biosensor that is provided in the main body and includes a first contact surface contactable with the living body; wherein
the main body includes a first rigid portion that supports the first biosensor, and a deformable portion that supports the first rigid portion and is softer than the first rigid portion.

13. The biological information acquisition device according to claim 12, wherein the first biosensor includes a bio-acoustic sensor that includes a vibration plate including the first contact surface and a piezoelectric element to detect vibration of the vibration plate and to measure a sound emitted from the living body.

14. The biological information acquisition device according to claim 12, further comprising:
a second biosensor including a second contact surface contactable with the living body; wherein
the main body includes a second rigid portion that supports the second biosensor.

15. The biological information acquisition device according to claim 14, wherein
the second biosensor is an electrocardiographic sensor that includes a plurality of electrodes each including the second contact surface and operable to acquire an electrocardiographic waveform of the living body;
a plurality of the second rigid portions are provided; and
the plurality of electrodes of the second biosensor are provided in the plurality of second rigid portions.

16. The biological intonation acquisition device according to claim 15, wherein the first rigid portion is between the plurality of second rigid portions.

17. The biological information acquisition device according to claim 16, wherein the first rigid portion is spaced apart from each of the plurality of second rigid portions.

18. The biological intonation acquisition device according to claim 15, wherein each of the plurality of electrodes has a rectangular or circular shape.

19. The biological information acquisition device according to claim 15, wherein angular positions of the plurality of electrodes with respect to the first biosensor are different by 90 or 180 degrees.
